SEQUENCE LISTING (3 sequences)

SEQ ID NO: 1 - protein, 73 aa, Artificial Sequence
  The sequence is synthetized.
LIKGNFHAVYRDDLKKLLETECPQYIRKKGADVWFKELDINTDGAVNFQEFLILVIKMGVAAHKKSHEESHKE

SEQ ID NO: 2 - protein, 94 aa, Artificial Sequence
  The sequence is synthetized.
QYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 3 - protein, 171 aa, Artificial Sequence
  The sequence is synthetized.
LIKGNFHAVYRDDLKKLLETECPQYIRKKGADVWFKELDINTDGAVNFQEFLILVIKMGVAAHKKSHEESHKEGGGGQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

(19) United States
(12) Patent Application Publication
(10) Pub. No.: US 2022/0308067 A1
(43) Pub. Date: Sep. 29, 2022

(54) APPLICATIONS OF SERUM S100A8/A9 COMPLEX LEVEL IN PROGNOSIS ASSESSMENT OF ACUTE MYOCARDIAL INFARCTION

(71) Applicant: BEIJING INSTITUTE OF HEART LUNG AND BLOOD VESSEL DISEASES, Beijing (CN)

(72) Inventors: Jie DU, Beijing (CN); Yulin LI, Beijing (CN); Xinying YANG, Beijing (CN); Ping LI, Beijing (CN)

(73) Assignee: BEIJING INSTITUTE OF HEART LUNG AND BLOOD VESSEL DISEASES, Beijing (CN)

(21) Appl. No.: 17/268,480

(22) PCT Filed: Aug. 16, 2019
(86) PCT No.: PCT/CN2019/101102

§ 371 Date: Feb. 15, 2021

(30) Foreign Application Priority Data
Aug. 17, 2018 (CN) 201810939526.4

(51) Int. Cl. G01N 33/68; G01N 33/543

(52) U.S. Cl. CPC G01N 33/6893; G01N 33/54306; G01N 2800/56; G01N 2800/324

(57) ABSTRACT

Applications of a serum S100A8/A9 complex level in the prognosis assessment of acute myocardial infarction (AMI) are disclosed. The prognosis assessment refers to predicting short-term and long-term prognosis of an AMI patient undergoing percutaneous coronary intervention (PCI) and determining whether the patient falls into a high-risk group of postoperative adverse events (AEs) or a low-risk group of the postoperative AEs. The AEs include but are not limited to death, cardiogenic shock (CS), and acute heart failure (AHF). The S100A8/A9 complex is a heterodimer complex formed by S100A8 protein (Calgranulin A, MRP8) and S100A9 protein (Calgranulin B, MRP14) in a calcium ion-dependent manner. The S100A8/A9 complex refers to the S100A8/A9 complex in serum of the patient within 24 h after the PCI. An antibody pair for detecting the serum S100A8/A9 complex is also disclosed.

### CROSS REFERENCE TO THE RELATED APPLICATIONS

This application is the national phase entry of International Application No. PCT/CN2019/101102, filed on Aug. 16, 2019, which is based upon and claims priority to Chinese Patent Application No. 201810939526.4, filed on Aug. 17, 2018, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBCHTN002-PKG_Sequence Listing.txt, created on 01/21/2022, and is 3,641 bytes in size.

### TECHNICAL FIELD

The present invention belongs to the field of medical biotechnology, and specifically relates to applications of a serum S100A8/A9 complex level in the prognosis assessment of acute myocardial infarction (AMI).

### BACKGROUND

With the development of social economy, the change of people's lifestyles, and the acceleration of population aging, an incidence of cardiovascular diseases (CVDs) in China is on an upward trend. According to “Report on Cardiovascular Diseases in China, 2018”, there were 290 million patients with CVD in China, including 2.5 million patients with acute myocardial infarction (AMI). AMI is the most serious manifestation among coronary heart diseases (CHDs), which often can lead to a series of serious cardiovascular events and become life threatening. AMI mostly occurs based on coronary atherosclerotic stenosis. Coronary atherosclerotic plaques rupture due to some factors, and circulating platelets accumulate on the surface of ruptured plaques to form thrombi, which abruptly block a coronary lumen and thus cause myocardial ischemic necrosis. Therefore, early revascularization is the key to the treatment of AMI. The clinical practice of percutaneous coronary intervention (PCI) significantly reduces mortality rates among AMI patients at an acute phase, but some AMI patients, after undergoing PCI revascularization, are still subjected to malignant cardiovascular events such as cardiogenic shock (CS) and acute heart failure (AHF) due to ischemia-reperfusion injury (RI). Therefore, there is an urgent need to preemptively identify such patients at a high risk of IRI and give timely intervention to reduce the incidence of intra-hospital and long-term adverse events (AEs).

In recent years, there have been more and more studies on prognostic markers of AMI. The most common ones include the following: troponin, N-terminal pro-B-type natriuretic peptide (NT-proBNP), growth differentiation factor 15 (GDF15), C-reactive protein (CRP), etc. When myocardium is damaged, troponin is released into the blood. With the characteristics of high specificity, high sensitivity, or the like, troponin is mainly used for the early clinical diagnosis of AMI and also shows prediction value for short-term and long-term prognosis of AMI patients. NT-proBNP is produced by cardiomyocytes. When the tension and load of ventricles increase, an NT-proBNP concentration in the circulation increases. Studies have shown that an NT-proBNP level is related to left ventricular remodeling after AMI and can be used to predict the occurrence of short-term and long-term cardiovascular AEs, but it is susceptible to factors such as gender, age, hypertension, and past myocardial infarction (MI) history. Existing markers mainly reflect non-specific pathological mechanisms after AMI such as neutrophil and endotheliocyte activation, inflammation, and cardiomyocyte necrosis, and cannot reflect the pathological change of IRI.

S100A8 protein (Calgranulin A, MRP8) and S100A9 protein (Calgranulin B, MRP14) are members of the S100 family of calcium-binding proteins (CaBP), which often form a heterodimer in a calcium ion-dependent manner, namely, a protein complex S100A8/A9 (hereinafter referred to as S100A8/A9). As a pro-inflammatory cytokine, S100A8/A9 accounts for about 40% of all cytoplasmic proteins in neutrophils, and is released rapidly after activation. Monocytes, macrophages, endotheliocytes, and platelets all can produce a small amount of S100A8/A9.

According to previous studies, dynamic transcriptomics in mouse cardiac IRI models shows that S100A8/A9 is significantly upregulated at an early stage of reperfusion. As an inflammatory cytokine, S100A8/A9 down-regulates the expression of NDUFs gene by inhibiting TLR4/Erk-mediated PGC-1α/NRF1 signal transduction and thus inhibits the function of mitochondrial complex I to cause mitochondrial dysfunction, ultimately leading to cardiomyocyte death. The administration of an S100A9 neutralizing antibody can significantly reduce myocardial IRI and improve cardiac functions.

Therefore, from the perspective of effective and rapid identification of high-risk patients in clinics, S100A8/A9 expression levels at multiple time points before and after PCI treatment of AMI patients are detected, and detection results show that S100A8/A9, as an inflammatory cytokine involved in the pathophysiological process of IRI, may be used to predict the occurrence of intra-hospital cardiovascular AEs after PCI and to effectively evaluate the long-term prognosis.

### SUMMARY

Based on the above-mentioned existing clinical problems, the present invention studies a relationship of a serum S100A8/A9 complex level with symptoms and a prognosis of an AMI patient. In the present invention, 207 healthy people and 208 AMI patients are selected, the ELISA technology is used to quantify serum S100A8/A9 complex levels in the healthy people and AMI patients, and the same method is used to quantify serum S100A8/A9 complex levels in AMI patients before and after PCI. Statistical results show that the protein complex can be used as a marker to distinguish AMI patients from healthy people and can also be used as a predictor of AEs in AMI patients after PCI. Compared with existing clinical testing products, the marker can better indicate AMI patients prone to short-term and long-term AEs.

The present invention relates to an application of an S100A8/A9 complex in a prediction of short-term and long-term prognosis of an AMI patient undergoing PCI as a serological diagnostic marker.

The S100A8/A9 complex is a heterodimer complex formed by S100A8 protein (Calgranulin A, MRP8) and S100A9 protein (Calgranulin B, MRP14) in a calcium ion-dependent manner. Preferably, the S100A8/A9 complex may refer to an S100A8/A9 complex in serum of the patient within 24 h after PCI.

The short-term prognosis and long-term prognosis may refer to a prognosis during an intra-hospital period after PCI and a prognosis 6 months or longer after PCI, respectively.

The prognosis refers to a probability of a postoperative adverse event (AE) in the patient, and the AE includes but is not limited to death, CS, and AHF.

The present invention also relates to an application of the S100A8/A9 complex in a preparation of a test kit for predicting short-term and long-term prognosis of an AMI patient undergoing PCI.

The predicting short-term and long-term prognosis of the AMI patient undergoing PCI refers to predicting a probability of a postoperative AE in the patient, and the AE includes but is not limited to death, CS, and AHF.

The short-term prognosis and long-term prognosis may refer to a prognosis during an intra-hospital period after PCI and a prognosis 6 months or longer after PCI, respectively.

The S100A8/A9 complex is a heterodimer complex formed by S100A8 protein (Calgranulin A, MRP8) and S100A9 protein (Calgranulin B, MRP14) in a calcium ion-dependent manner. Preferably, the S100A8/A9 complex may refer to an S100A8/A9 complex in serum of the patient within 24 h after PCI.

The present invention also relates to a test kit including a detection reagent for detecting the S100A8/A9 complex, and the detection reagent includes but is not limited to:

(1) an antibody that specifically binds to the S100A8/A9 complex, including but not limited to a polyclonal antibody (pAb), a monoclonal antibody (mAb), a single-chain variable fragment (scFv), a functional antibody fragment, an antibody Fab region, a nanobody, a chimeric antibody, and a multispecific antibody;

(2) a ligandin or polypeptides that specifically bind to the S100A8/A9 complex; and

(3) a non-protein compound that specifically recognizes the S100A8/A9 complex.

Preferably, the test kit may be:

(1) an enzyme-linked immunosorbent assay (ELISA) kit;

(2) a colloidal gold test strip assay kit;

(3) a chemiluminescent assay kit; and

(4) a flow cytometry (FCM) assay kit.

The S100A8/A9 complex is a heterodimer complex formed by S100A8 protein (Calgranulin A, MRP8) and S100A9 protein (Calgranulin B, MRP14) in a calcium ion-dependent manner.

The present invention also relates to an application of the S100A8/A9 complex in a prediction of short-term and long-term prognosis of an AMI patient undergoing PCI. The prediction of short-term and long-term prognosis of the AMI patient undergoing PCI refers to determining whether the patient falls into a high-risk group of a postoperative AE (short-term and long-term) or a low-risk group of the postoperative AE (short-term and long-term), and the AE includes but is not limited to death, CS, and AHF.

The short-term prognosis and long-term prognosis may refer to a prognosis during an intra-hospital period after PCI and a prognosis 6 months or longer after PCI, respectively:

the high-risk group for the prognosis during the intra-hospital period may have an AE incidence of about 68% after PCI;

the low-risk group for the prognosis during the intra-hospital period may have an AE incidence of about 21% after PCI;

the high-risk group for the long-term prognosis may have an AE incidence of about 23% after PCI;

the low-risk group for the long-term prognosis may have an AE incidence of about 6% after PCI;

the high-risk group of the postoperative AE (short-term and long-term) is determined when a serum S100A8/A9 complex expression level in the patient 24 h after PCI is greater than or equal to 4,540 ng/ml; and

the low-risk group of the postoperative AE (short-term and long-term) is determined when the serum S100A8/A9 complex expression level in the patient 24 h after PCI is less than 4,540 ng/ml.

The S100A8/A9 complex is a heterodimer complex formed by S100A8 protein (Calgranulin A, MRP8) and S100A9 protein (Calgranulin B, MRP14) in a calcium ion-dependent manner.

The present invention also relates to an application of the S100A8/A9 complex in a preparation of a test kit for predicting a prognosis of an AMI patient undergoing PCI. The predicting short-term and long-term prognosis of the AMI patient undergoing PCI refers to determining whether the patient falls into a high-risk group of a postoperative AE (short-term and long-term) or a low-risk group of the postoperative AE (short-term and long-term), and the AE includes but is not limited to death, CS, and AHF.

The short-term prognosis and long-term prognosis may refer to a prognosis during an intra-hospital period after PCI and a prognosis 6 months or longer after PCI, respectively;

the high-risk group for the prognosis during the intra-hospital period may have an AE incidence of about 68% after PCI;

the low-risk group for the prognosis during the intra-hospital period may have an AE incidence of about 21% after PCI;

the high-risk group for the long-term prognosis may have an AE incidence of about 23% after PCI;

the low-risk group for the long-term prognosis may have an AE incidence of about 6% after PCI;

the high-risk group of the postoperative AE (short-term and long-term) is determined when a serum S100A8/A9 complex expression level in the patient 24 h after PCI is greater than or equal to 4,540 ng/ml; and

the low-risk group of the postoperative AE (short-term and long-term) is determined when the serum S100A8/A9 complex expression level in the patient 24 h after PCI is less than 4,540 ng/ml.

The S100A8/A9 complex is a heterodimer complex formed by S100A8 protein (Calgranulin A, MRP8) and S100A9 protein (Calgranulin B, MRP14) in a calcium ion-dependent manner.

The test kit may also include a detection reagent for detecting the serum S100A8/A9 complex expression level, and the detection reagent includes but is not limited to:

(1) an antibody that specifically binds to the S100A8/A9 complex, including but not limited to a pAb, an mAb, an scFv, a functional antibody fragment, an antibody Fab region, a nanobody, a chimeric antibody, and a multispecific antibody;

(2) a ligandin or polypeptides that specifically bind to the S100A8/A9 complex; and

(3) a non-protein compound that specifically recognizes the S100A8/A9 complex.

The test kit may be:

(1) an ELISA kit;

(2) a colloidal gold test strip assay kit;

(3) a chemiluminescent assay kit; and

(4) an FCM assay kit.

### BRIEF DESCRIPTION OF THE DRAWINGS

FIG. 1 is a diagram showing the comparison of serum S100A8/A9 complex levels in healthy people with that in AMI patients;

FIG. 2 is a diagram showing the detection results of S100A8/A9 complex expression levels in AMI patients before and after PCI treatment;

FIG. 3 is a diagram showing serum S100A8/A9 complex levels of patients in (major adverse cardiovascular event) MACE group and Non-MACE group before and 1 day after PCI,

FIG. 4 is a diagram showing a receiver operating characteristic (ROC) curve for predicting the occurrence of intra-hospital MACEs in AMI patients after PCI from a difference between S100A8/A9 complex expression levels in AMI patients before and after PCI;

FIG. 5 is a diagram showing long-term MACE survival curves of AMI patients after PCI, with a difference value of 2,248.13 ng/ml between S100A8/A9 complex expression levels in AMI patients before and after PCI as a cut-off value;

FIG. 6 is a diagram showing serum S100A8/A9 expression levels in healthy people and AMI patients before and after PCI treatment;

FIG. 7 is a diagram showing serum S100A8/A9 expression levels before and after PCI in patients of MACE group and Non-MACE group with long-term prognosis follow-up;

FIG. 8 is a diagram showing an ROC curve for predicting the occurrence of MACEs in AMI patients during long-term follow-up after PCI based on serum S100A8/A9 complex expression levels in AMI patients within 24 h after PCI;

FIG. 9 is a diagram showing MACE survival curves of AMI patients during long-term follow-up after PCI, with a serum S100A8/A9 complex expression level of 4,540 ng/ml in AMI patients within 24 h after PCI as a cut-off value:

FIG. 10 is a diagram showing serum S100A8/A9 expression levels at 5 time points before and after PCI in AMI patients of two groups obtained by grouping the patients according to whether there are intra-hospital AEs;

FIG. 11 is a diagram showing serum S100A8/A9 expression levels at 2 time points before and after PCI in patients of two groups obtained by grouping 349 AMI patients in validation cohort according to whether there are intra-hospital AEs;

FIG. 12 is a diagram showing Kaplan-Meier survival curves for MACEs verified in 559 (210+349) AMI patients, with a set cut-off value of 4,540 ng/ml);

FIG. 13A is a diagram showing S100A8 antigen-antibody titer detection results, and FIG. 13B is a diagram showing an S100A8 antigen-antibody pairing curve;

FIG. 14A is a diagram showing S100A9 antigen-antibody titer detection results, and FIG. 14B is a diagram showing an S100A9 antigen-antibody pairing curve; and

FIG. 15 is a diagram showing an S100A8/A9 antigen-antibody pairing curve.

### DETAILED DESCRIPTION OF THE EMBODIMENTS

### Example 1: Difference in the S100A8/A9 Expression Levels Between AMI Patients and Healthy People

According to the principle of gender and age matching, serum samples of 207 healthy people and 208 AMI patients were selected, and an S100A8/A9 expression levels were detected by ELISA. An S100A8/A9 expression level in AMI patients was 1.91 times higher than that in healthy people (P<0.001).

I. Experimental Steps:

Kit: R&D Systems, Inc, Human S100A8/S100A9, Heterodimer Immunoassay

(1) Preparation of Reagents:

1. All reagents were equilibrated to room temperature before use.

2. Wash buffer: If crystals were formed in a concentrate, the concentrate should be equilibrated to room temperature and shaken gently until the crystals were completely dissolved, and ionized water or distilled water was added to dilute a wash buffer from 20 ml to 500 ml.

3. Substrate solution: Chromogenic reagents A and B should be mixed in equal volumes 15 min before use and stored in the dark. Each well required 200 ul of a mixture of chromogenic reagents A and B in equal volumes.

4. S100A8/A9 standard: An S100A8/A9 standard was re-prepared using calibrator diluent RD5-10. A re-prepared product was a 40 ng/ml stock solution. The standard was gently stirred for at least 15 min before diluted.

5. 250 ul of appropriate calibrator diluent RD5-10 was pipetted into each tube. The stock solution was used to prepare a series of dilutions (20 ng/ml, 10 ng/ml, 5 ng/ml, 2.5 ng/ml, 1.25 ng/ml, and 0.625 ng/ml). An undiluted standard (40 ng/ml) was adopted as a high standard, and a calibrator diluent was adopted as a 0 standard (0 pg/ml).

(2) Determination Steps:

Before use, all reagents and samples were equilibrated to room temperature, and all samples, standards and controls were determined in duplicate.

1. All reagents and working standards were prepared, and serum samples were diluted by 150 times.

2. Excess microplates were removed and put back into a tin foil bag with desiccant, which was resealed.

3. 50 ul of assay diluent RD1-34 was added to each hole.

4. 50 ul of each of standards, samples, and controls was added to each well. The plates were sealed with rubber strips and incubated at room temperature for 2 h, and then distributions of determined standards and samples were recorded.

5. Liquid in each well was pipetted away, and then each well was fully washed with 400 ul of wash buffer to completely remove the liquid and patted dry on clean paper. The washing was conducted 4 times.

6. 200 ul of S100A8/A9 conjugate was added to each well, and then plates were sealed with new rubber strips and warmed at room temperature for 2 h.

7. The step 5 was repeated.

8. 200 ul of the substrate solution was added to each well, and a resulting mixture was incubated for 30 min at room temperature in the dark.

9. 50 ul of a stop solution was added to each well, and a color in the well should change from blue to yellow. If the color in the well is green or if the color changes unevenly, the plate should be patted gently to ensure thorough mixing.

10. A microplate reader was used to determine the absorbance of each well at 450 nm within 30 min. If wavelength calibration is effective, 540 nm or 570 nm is set. If wavelength calibration is unavailable, a reading at 540 nm or 570 nm is subtracted from a reading at 450 nm. This method can correct optical defects of a plate. Reading directly at 450 nm without correction can be either slightly-high or slightly-low.

(3) Calculation Results:

1. An OD value of the zero standard was subtracted from an OD value of each of the standards, controls, and samples, and an average was taken for two replicate wells.

2. With a 4-parameter curve, a standard curve was established by computer software.

3. If a sample has been diluted, a concentration obtained from the standard curve must be multiplied by a dilution factor.

II. Experimental Results:

Results were shown in FIG. 1 and Table 1 below. Compared with healthy people, serum S100A8/A9 levels in AMI patients significantly increased.

TABLE 1
Serum S100A8/A9 complex levels in healthy people and AMI patients

| | Healthy control (HC) | AMI patients |
|---|---|---|
| Detection index | (n = 207) | (n = 208) |
| S100A8/A9, ng/ml | 1,723 ± 193.7 | 3,295 ± 200.4 |
| (mean ± SEM) | | |

### Example 2: Expression of S100A8/A9 in 210 AMI Patients at Two Consecutive Time Points (Before and 1 Day after PCI) in Hospital

Samples were collected from 210 AMI patients at two consecutive time points (before PCI (day 0) and 1 day after PCI (day 1)), and S100A8/A9 complex expression level in serum of patients were determined by the same ELISA experiment as in Example 1.

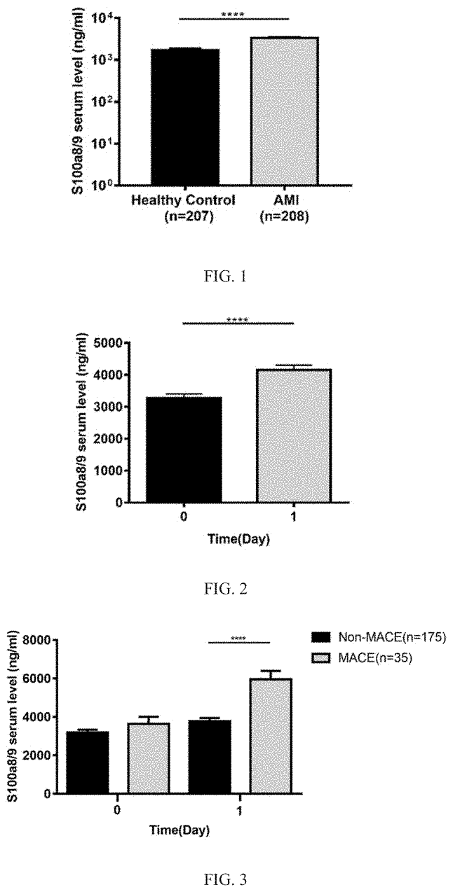

1. Detection results of S100A8/A9 expression levels before and after PCI were shown in FIG. 2 and Table 2. The results showed that an S100A8/A9 expression level after PCI was higher than that before PCI (P<0.001).

TABLE 2
Serum S100A8/A9 complex levels in AMI patients before and after PCI

| | Before PCI | 1 day after PCI |
|---|---|---|
| Detection index | (n = 210) | (n = 210) |
| S100A8/A9, ng/ml | 3,270 ± 134.2 | 4,151 ± 156.9 |
| (mean ± SEM) | | |

### Example 3: Prognosis Assessment Value of S100A8/A9 for Intra-Hospital MACEs

According to the occurrence of intra-hospital MACEs (death and AHF), 210 AMI patients were divided into an MACE group (35 patients) and a non-MACE group (175 patients). S100A8/A9 complex expression levels in serum of patients were determined by the same ELISA experiment as in Example 1.

Statistical Results:

(1) S100A8/A9 complex expression difference between the MACE group and the non-MACE group before PCI (day 0) and 1 day after PCI (day 1): 1 day after PCI, there was a statistically significant difference between the two groups, and the MACE group was 1.57 times higher than the non-MACE group (P<0.001), as shown in FIG. 3 and Table 3.

TABLE 3
Serum S100A8/A9 complex levels of patients in the MACE
group and the non-MACE group before and after PCI

| | Before PCI | | 1 day after PCI | |
|---|---|---|---|---|
| | MACE | Non-MACE | MACE | Non-MACE |
| Detection index | (n = 35) | (n = 175) | (n = 35) | (n = 175) |
| S100A8/A9, ng/ml | 3,633 ± | 3,197 ± | 5,960 ± | 3,789 ± |
| (mean ± SEM) | 375.9 | 142.4 | 437.8 | 153.3 |

(2) A logistic regression model was established to illustrate a relationship between marker levels and intra-hospital MACEs. In order to reflect the fluctuation of S100A8/A9 levels, according to marker levels at two time points (before PCI (day 0) and 1 day after PCI (day 1)), the following marker level combinations were obtained: Δ 1d (a difference between a marker level 1 day after PCI and a marker level before PCI), Δ 1d+1d (a sum of Δ 1d with a marker level 1 day after PCI), Δ 1d/0d (a ratio of Δ 1d to a marker level before PCI), and SD (a standard deviation for marker levels at the two time points). A logistic regression model was established to correct the eight factors of age, gender, time to open blood vessels, hypertension history, diabetes history, hyperlipemia history, smoking history, and infarction location. The 6 kinds of marker levels (before PCI, 1 day after PCI, Δ 1d, Δ 1d+1d, Δ 1d/0d, and SD) all have independent prediction value for intra-hospital MACEs and are superior to high-sensitivity cardiac troponin I (hs-cTnI).

TABLE 4
A relationship between marker levels and intra-hospital
MACEs according to a logistic regression model

| | Unadjusted OR (95% CI) | Adjusted OR (95% CI) |
|---|---|---|
| Before PCI (day 0) | 1.642 (1.048-2.573) | 1.800 (1.097-2.952) |
| | P = 0.031 | P = 0.020 |
| 1 day after PCI (day 1) | 3.009 (1.898-4.770) | 3.988 (2.301-6.912) |
| | P < 0.001 | P < 0.001 |
| Δ 1 d | 2.422 (1.536-3.820) | 2.624 (1.597-4.310) |
| | P < 0.001 | P < 0.001 |
| Δ 1 d + 1 d | 3.420 (2.114-5.532) | 4.098 (2.375-7.071) |
| | P < 0,001 | P < 0.001 |
| Δ 1 d/0 d | 1.830 (1.171-2.858) | 1.878 (1.176-2.999) |
| | P < 0.001 | P = 0.008 |
| SD | 2.165 (1.431-3.276) | 2.450 (1.540-3.895) |
| | P < 0.001 | P < 0.001 |
| Baseline hs-cTnI | 0.866 (0.477-1.571) | 0.703 (0.372-1.329) |
| | P = 0.635 | P = 0.279 |
| Peak hs-cTnI | 1.524 (1.085-2.141) | 1.557 (1.077-2.252) |
| | P = 0.015 | P = 0.019 |

(Unadjusted OR refers to inputting only a marker level into the logistic regression model. Adjusted OR refers to inputting a marker level and an aforementioned correction factor into the logistic regression model. As there are 8 markers, correction needs to be conducted 8 times)

### Example 4: Prognosis Assessment Value of S100A8/A9 for Long-Term MACEs

Among the 210 AMI patients, 4 died in the hospital, so the remaining 206 patients were followed up for a long term. A median follow-up time was 21.7 (IQR: 13.7 to 29.6) months. 15 people underwent MACEs (who were subjected to all-cause death or re-admitted to hospital due to heart failure).

Experimental Statistical Results:

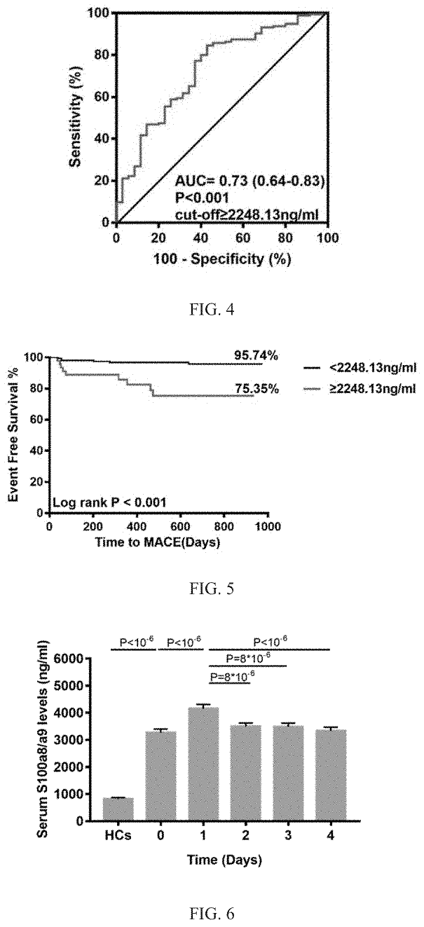

(1) In the intra-hospital MACE analysis, it was found that the Δ 1d S100A8/A9 level exhibited the best prognosis assessment value. The ROC curve analysis was used to determine a cut-off value for S100A8/A9 Δ 1d intra-hospital AE prediction. A specific ROC curve was shown in FIG. 4, and ROC data therein were shown in Table 5 below.

TABLE 5

AUC = 0.732 (0.640-0.825)
P < 0.001
cut-off: ≥2,248.13 ng/ml
Sensitivity: 57.14% (39.4-73.7)
Specificity: 84.57% (78.4-89.6)

(2) The Kaplan-Meier survival curve was used to evaluate a prediction value of the cut-off value for S100A8/A9 Δ 1d intra-hospital MACE prediction on long-term AEs. Patients with a marker level <2,248.13 ng/ml had better long-term prognosis than those with a higher marker level (P<0.001) (survival curves for the two groups of patients were shown in FIG. 5).

### Example 5: Expression of S100A8/A9 in 210 AMI Patients at 5 Consecutive Time Points in the Hospital (Before PCI, 1 Day after PCI, 2 Days after PCI, 3 Days after PCI, and 4 Days after PCI), and in 425 Healthy People (HCs)

Serum samples were collected from 210 AMI patients at 5 consecutive time points (before PCI (day 0), 1 day after PCI (day 1), 2 days after PCI (day 2), 3 days after PCI (day 3), and 4 days after PCI (day 4)) and from 425 healthy people (healthy controls, HCs), and S100A8/A9 expression levels were determined by ELISA. An S100A8/A9 expression level in AMI patients was higher than that in healthy people (P<0.001). An S100A8/A9 expression level 1 day after PCI was 1.27 times higher than that before PCI (P<0.001) and then gradually decreased.

Experimental results were shown in Table 6 and FIG. 6 below. It can be seen that, among all AMI patients undergoing PCI, a serum sample collected 1 day after PCI had a significantly increased overall S100A8/A9 complex level compared with serum samples collected at the other three detection time points (2 to 4 days after PCI).

TABLE 6
S1000A8/A9 complex levels in serum

| Group | S100A8/A9, ng/ml (mean ± SEM) |
|---|---|
| HCs | 820.8 ± 58.54 |
| Before PCI | 3,270 ± 134.2 |
| 1 day after PCI | 4,151 ± 156.9 |
| 2 days after PCI | 3,500 ± 128.8 |
| 3 days after PCI | 3,488 ± 127.9 |
| 4 days after PCI | 3,331 ± 138.9 |

Notes:
After a sample set of healthy people is added and the detection method is corrected, a serum S100A8/A9 complex level in healthy people is slightly different from data in Example 1.

After repeated verification, the data in Table 6 are very close to real data.

### Example 6: Prognosis Assessment Value of S100A8/A9 for Long-Term MACEs

Further long-term follow-up was conducted on the aforementioned 210 patients, and a median follow-up time was increased to 789 (IQR: 460 to 1,034) days. The long-term MACEs in the Example 4 were re-examined and distinguished, and incidental suspected MACEs in short-term follow-up were excluded. 24 people were confirmed to have cardiovascular AEs (MACE, who were subjected to all-cause death or CS, or re-admitted to hospital due to heart failure).

Given the comprehensiveness of MACEs, CS was also counted as MACE when it was counted again.

Experimental Results:

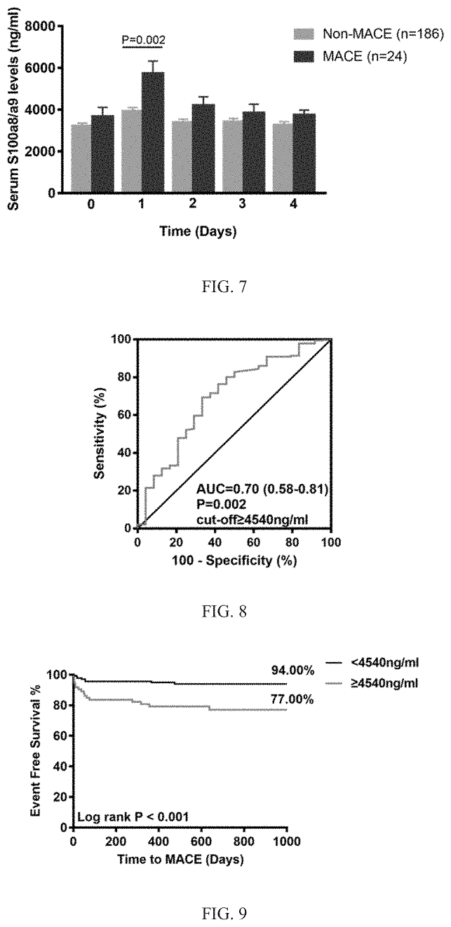

(1) S100A8/A9 expression difference between the MACE group and the non-MACE group before PCI (day 0), 1 day after PCI (day 1), 2 days after PCI (day 2), 3 days after PCI (day 3), and 4 days after PCI (day 4). Only 1 day after PCI, there was a statistically significant difference between S100A8/A9 levels in serum samples of patients in the two groups, and the MACE group was 1.45 times higher than the non-MACE group (P=0.002). Comparative data of S100A8/A9 levels in serum samples obtained according to the grouping situation were shown in Table 7 and FIG. 7 below.

TABLE 7
Correlation between serum S100A8/A9
complex levels and long-tern MACEs

| | Non-MACE (n = 186) | MACE (n = 24) |
|---|---|---|
| | S100A8/A9, ng/ml | S100A8/A9, ng/ml |
| Group | (mean ± SEM) | (mean ± SEM) |
| Before PCI | 3,218 ± 140.7 | 3,673 ± 435.4 |
| 1 day after PCI | 3,946 ± 154.5 | 5,740 ± 588.3 |
| 2 days after PCI | 3,408 ± 134.6 | 4,207 ± 407.9 |
| 3 days after PCI | 3,439 ± 135.1 | 3,864 ± 395.2 |
| 4 days after PCI | 3,277 ± 153.8 | 3,750 ± 225.2 |

(2) A COX regression model was established to illustrate a relationship between marker levels and long-term MACEs. According to marker levels at five time points (before PCI (day 0), 1 day after PCI (day 1), 2 days after PCI (day 2), 3 days after PCI (day 3), and 4 days after PCI (day 4), a COX regression model was established to correct TIMI risk scores. Results showed that an S100A8/A9 level 1 day after PCI exhibited the best prediction value for long-term MACEs.

TABLE 8
Corrected S100A8/A9 value 1 day after PCI has the
best prediction value for long-term MACEs

| | Unadjusted HR | Adjusted HR |
|---|---|---|
| Group | (95% CI) | (95% CI) |
| Before PCI (day 0) | 1.283 (0.789-2.086) | 0.959 (0.529-1.739) |
| | P = 0.315 | P = 0.891 |
| 1 day after PCI (day 1) | 2.067 (1.317-3.243) | 2.039 (1.149-3.618) |
| | P = 0.002 | P = 0.015 |
| 2 days after PCI (day 2) | 1.515 (1.000-2.297) | 1.165 (0.681-1.993) |
| | P = 0.050 | P = 0.578 |
| 3 days after PCI (day 3) | 1.360 (0.886-2.089) | 0.960 (0.576-1.603) |
| | P = 0.159 | P = 0.877 |
| 4 days after PCI (day 4) | 1.392 (0.899-2.155) | 0.833 (0.441-1.574) |
| | P = 0.138 | P = 0.574 |

In the long-term MACE analysis, it was found that the S100A8/A9 level 1 day after PCI exhibited the best prognosis assessment value. Therefore, in order to achieve the purpose of clinical use, a cut-off value was found to accurately achieve risk stratification for patients.

Experimental Results:

(1) The ROC curve analysis was used to determine a cut-off value for long-term AE prediction using S100A8/A9 1 day after PCI.

Results were shown in FIG. 8. Values determined by ROC curve analysis were as follows:

AUC=0.70 (0.58-0.81), P=0.002

cut-off ≥4,540 ng/ml

Sensitivity: 66.7%

Specificity: 69.4%

(2) The Kaplan-Meier survival curve was used to evaluate the prediction value of the S100A8/A9 expression level 1 day after PCI for long-term MACEs. Patients with a marker level <4,540 ng/ml had better long-term prognosis than those with a higher marker level (FIG. 9) (P<0.001).

### Example 7: Prognosis Re-Assessment of S100A8/A9 on Intra-Hospital Cardiovascular AEs

The occurrence of intra-hospital (the intra-hospital refers to a period from the end of PCI to discharge from hospital) cardiovascular AEs (MACE: all-cause death, CS, AHF) in 210 AMI patients was observed and counted again. A total of 32 patients underwent MACEs.

Given the comprehensiveness of MACEs, CS was also counted as MACE when it was counted again.

Experimental Results:

(1) S100A8/A9 expression difference between the MACE group and the non-MACE group before PCI (day 0), 1 day after PCI (day 1), 2 days after PCI (day 2), 3 days after PCI (day 3), and 4 days after PCI (day 4). There was a statistically significant difference between the two groups at each time point, and there was the most significant difference 1 day after PCI (as shown in Table 7 and FIG. 10 below). The MACE group was 1.69 times higher than the non-MACE group (P<0.001)

TABLE 7
Comparison of S100A8/A9 complex levels in serum of patients
in the MACE group/non-MACE groups in the hospital

| S100A8/A9, ng/ml | Non-MACE | MACE |
|---|---|---|
| (mean ± SEM) | (n = 178) | (n = 32) |
| Before PCI | 3,102 ± 156.5 | 3,907 ± 444.1 |
| 1 day after PCI | 3,601 ± 153.7 | 6,075 ± 451.4 |
| 2 days after PCI | 3,262 ± 136.3 | 4,946 ± 430.2 |
| 3 days after PCI | 3,247 ± 137.5 | 5,033 ± 405 |
| 4 days after PCI | 3,206 ± 240.6 | 4,611 ± 557.4 |

(2) According to marker levels at five time points (before PCI (day 0), 1 day after PCI (day 1), 2 days after PCI (day 2), 3 days after PCI (day 3), and 4 days after PCI (day 4), a COX regression model was established to correct TIMI risk scores. An S100A8/A9 level 1 day after PCI exhibited the best prediction value for intra-hospital MACEs (as shown in Table 8 below).

TABLE 8
Corrected and uncorrected prediction values

| | Unadjusted HR | Adjusted HR |
|---|---|---|
| | (95% CI) | (95% CI) |
| Before PCI (day 0) | 1.693 (1.098-2.613) | 1.722 (1.121-2.644) |
| | P = 0.017 | P = 0.013 |
| 1 day after PCI (day 1) | 3.142 (1.977-4.994) | 2.932 (1.871-4.595) |
| | P < 0.001 | P< 0.001 |
| 2 days after PCI (day 2) | 2.007 (1.349-2.986) | 1.946 (1.303-2.908) |
| | P = 0.001 | P = 0.001 |
| 3 days after PCI (day 3) | 2.115 (1.388-3.223) | 2.165 (1.401-3.345) |
| | P < 0.001 | P = 0.001 |
| 4 days after PCI (day 4) | 1.770 (1.055-2.969) | 1.785 (1.040-3.063) |
| | P = 0.031 | P = 0.036 |

The occurrence of intra-hospital cardiovascular AEs (MACE: all-cause death, CS, and AHF) in 349 AMI patients (not the aforementioned 210 patients, here 349 were newly enrolled patients) was observed, and a total of 47 patients underwent MACEs.

Experimental Results:

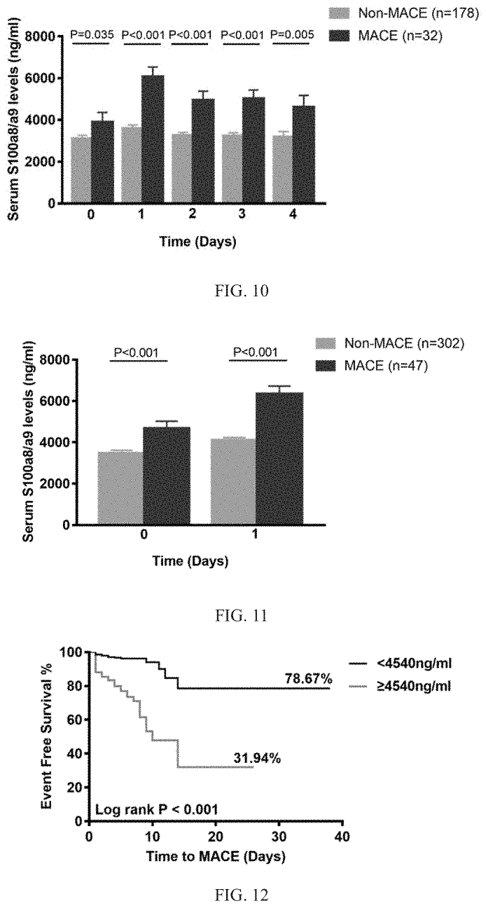

(1) S100A8/A9 expression difference between the MACE group and the non-MACE group before PCI (day 0) and 1 day after PCI (day 1) (as shown in Table 9 and FIG. 11 below). There was a statistically significant difference between the two groups at each time point, and there was the most significant difference 1 day after PCI. The MACE group was 1.55 times higher than the non-MACE group (P<0.001).

TABLE 9
Comparison of S100A8/A9 complex levels in serum of patients
in the MACE group/non-MACE group in the hospital

| S100A8/A9, ng/ml | Non-MACE | MACE |
|---|---|---|
| (mean ± SEM) | (n = 302) | (n = 47) |
| Before PCI | 3,476 ± 134.3 | 4,681 ± 339.1 |
| 1 day after PCI | 4,107 ± 123.5 | 6,358 ± 361.6 |

(2) According to marker levels at two time points (before PCI (day 0) and 1 day after PCI (day 1)), a COX regression model was established to correct TIMI risk scores (Table 10). Results showed that an S100A8/A9 level 1 day after PCI still exhibited independent prediction value for intra-hospital MACEs.

TABLE 10
Corrected and uncorrected prediction values

| | Unadjusted HR | Adjusted HR |
|---|---|---|
| | (95% CI) | (95% CI) |
| Before PCI (day 0) | 1.639 (1.190-2.258) | 1.573 (1.155-2.142) |
| | P = 0.002 | P = 0.004 |
| 1 day after PCI (day 1) | 2.422 (1.717-3.416) | 2.222 (1.569-3.147) |
| | P < 0.001 | P < 0.001 |

(3) With the cut-off value determined in Example 6 as a determination basis, a prediction value of the serum S100A8/A9 complex expression level 1 day after PCI to risk stratification for the occurrence of intra-hospital MACEs in AMI patients was verified. Data of a total of 559 patients enrolled in the two times were combined. Patients with an S100A8/A9 expression level 1 day after PCI <4,540 ng/ml were classified into a low-risk group, and patients with a level ≥4,540 ng/ml were classified into a high-risk group. The Kaplan-Meier survival curve was used for analysis, and results were shown in FIG. 12. It is shown that patients with a marker level <4,540 ng/ml had better intra-hospital prognosis than those with a higher marker level (P<0.001), suggesting that the cut-off value also has application value in the prediction of intra-hospital MACEs.

### Example 8: Preparation of Mouse Anti-S100A8, S100A9, and S100A8/A9 mAbs and Double-Antibody Sandwich ELISA

1. General mAb Preparation Method

1) Establishment of Hybridoma Cell Lines and Identification of mAb Subtypes

a. An immune procedure adopted 4 basic immunizations and 1 booster immunization. 2 healthy female BALB/c mice who were about 6 to 8 weeks old were selected and subjected to adaptive feeding for 1 week, and negative blood was collected as a control.

b. A medium-range immunization program (0.3 mL/mouse, 2 weeks/time) was adopted. For the first immunization (50 μg/mouse), an immunogen was mixed with a Freund's complete adjuvant (FCA) in equal volumes, and a resulting mixture was stirred and emulsified and then injected subcutaneously at multiple sites on the back. Thereafter, an immunogen was mixed with an FCA in equal volumes, and a resulting mixture was stirred and emulsified for routine immunization.

c. In the third immunization, usually, 50 μg of an antigen was mixed with TiterMax in equal amounts, and a resulting mixture was emulsified and then injected at multiple sites on the back. Titer was measured 7 days later. An injection without an adjuvant was prepared for booster immunization, which was injected at a dosage of 50 μg. 3 days after booster immunization, eyeballs were taken off for blood sampling, and serum was isolated and stored. Moreover, a spleen was collected for fusion.

d. During cell fusion, spleen cells and myeloma cells were mixed at a ratio of about 4:1 and then subjected to fusion under the pro-fusion action of polyethylene glycol (PEG, with a molecular weight of 1,450). Fused cells were cultivated in an HAT selective medium for 10 days, then positive hybridoma cells capable of reacting with a target protein were screened out by the indirect ELISA method, and the positive hybridoma cells obtained from preliminary screening were further cultivated for expansion. Two days later, His-tag hybridoma cells were excluded to re-screen out hybridoma cells targeting the target protein rather than the tag.

e. The limiting dilution method was used to consecutively subclone obtained positive hybridoma cells at least two times. Subclones obtained each time were cultivated with an HT selective medium. 8 to 10 days after subcloning, ELISA screening was conducted until monoclonal cells had a positive rate of 100% to obtain a monoclonal cell line capable of stably secreting an anti-target protein antibody.

f. A mouse mAb subtype identification chip kit (purchased from Raybiotech, USA) was used to identify an mAb subtype.

2) Preparation and Purification of Antibodies

a) Female healthy BALB/c mice who were 8 to 12 weeks old were selected and intraperitoneally injected with pristane at a dosage of 0.5 mL/mouse; and 7 to 10 days later, 1*10⁶ to 5*10⁶ monoclonal hybridoma cells were intraperitoneally injected into each mouse. It should be noted that PBS or serum-free medium needs to be used to make cells fall off or dilute cells.

b) An ascitic fluid was centrifuged at 10,000 r/min for 15 min, cell components and other precipitates, fat, oil layers, and so on were removed, and the intermediate layer was collected for determining antibody titer, dispensed, and stored at −70° C. for later use.

c) Precipitation using saturated ammonium sulfate (SAS): 5 mL of a treated ascitic fluid was pipetted to a small beaker, and 5.0 mL of PBS was filtered through a 0.22 μm filter membrane and then added dropwise to the beak under stirring; after a resulting solution was thoroughly mixed, 10 mL of an SAS solution (pH 7.4) was added, and a resulting mixture was further stirred slowly for 30 min, then stood for 2 h, and centrifuged at 10,000 r/min for 15 min; a resulting supernatant was removed, and a resulting precipitate was resuspended in PBS filtered through a 0.22 μm filter membrane; and a resulting suspension was filtered through a 0.22 μm filter membrane.

d) According to different antibody subtypes, different purification columns of GE Healthcare were selected to purify the antibodies, and obtained antibodies were dispensed and stored.

2. ELISA

In this example, S100A8, S100A9, and S100A8/A9 dimer were used as antigens and the antibody pairs prepared for the antigens in the previous example were used as recognition antibodies in the double-antibody sandwich to establish double-antibody sandwich ELISA for detecting target antigens.

1) Coating of ELISA Plate

An antibody 1 was diluted with a coating buffer (1 L in total: 1.5 g of Na₂CO₃, 2.9 g of NaHCO₃, 1.2 g of Na₂N₃, and the balance of ddH₂O; pH 9.6) to 1 μg/mL, a resulting solution was thoroughly mixed and added to a 96-well ELISA plate at 100 μL/well, and then the plate was sealed and placed overnight at 4° C.

2) Addition of to-be-Tested Antigens

To-be-tested antigens and negative control samples were added, separately; and then the plate was fully incubated, washed 6 times with a plate washer, patted dry, and stored at 4° C. for use or stored at −20° C. for a long time.

3) Detection antibodies were added at 100 μL/well, and the plate was incubated at 37° C., washed 6 times with a plate washer, and patted dry; then biotin-labeled goat anti-mouse IgG antibody (purchased from Raybiotech) with a specified concentration was added at 100 L/well, and the plate was incubated at 37° C. for 1 h and then washed; streptomycin-labeled horseradish peroxidase (HRP) was added at 100 μL/well, and the plate was incubated at 37° C. for 1 h and then washed; TMB substrate was added for chromogenic reaction; and after the chromogenic reaction was completed, 2 M concentrated sulfuric acid was added to stop the chromogenic reaction, and OD450 was measured with a microplate reader.

3. Sequence and Antibody Pairing Result for Each Antigen

1) The S100A8 antigen had a sequence shown in SEQ ID NO. 1:

LIKGNFHAVYRDDLKKLLETECPQYIRKKGADVWFKELDINTDGAVNFQE
FLILVIKMGVAAHKKSHEESHKE;

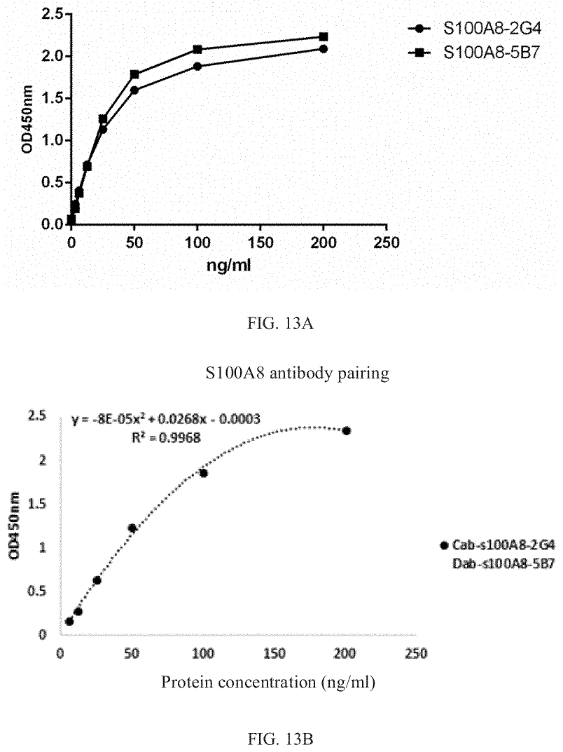

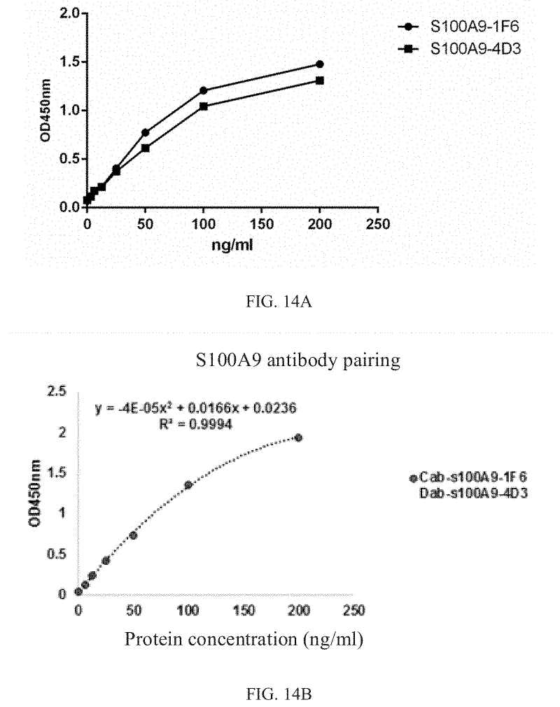

Antibody titer test results were shown in FIG. 13A and an antibody pairing curve was shown in FIG. 13B.

2) The S100A9 antigen had a sequence shown in SEQ ID NO. 2:

QYSVKLGHPDTLNQGEFKELNVRKDLQNFLKKENKNEKVIEHIMEDLDTN
ADKQLSFEEFIMLMARLTWASHEKMHEGDEGPGHHKPGLGEGTP;

Antibody titer test results were shown in FIG. 14A and an antibody pairing curve was shown in FIG. 14B.

3) The S100A8/A9 dimer antigen had a sequence shown in SEQ ID NO. 3:

LIKGNFHAVYRDDLKKLLETECPQYIRKKGADVWFKELDINTDGAVNFQE
FLILVIKMGVAAHKKSHEESHKEGGGGQYSVKLGHPDTLNQGEFKELVRK
DLQNFLKKENKNEKVIEHIMEDLDTNADKQLSFEEFIMLMARLTWASHEK
MHEGDEGPGHHHKPGLGEGTP;

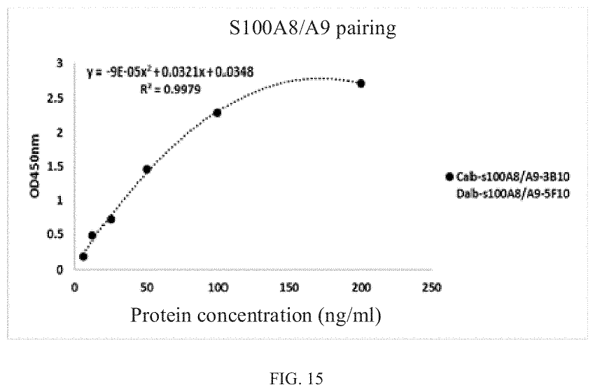

An antibody pairing curve was shown in FIG. 15.

Finally, it should be noted that the above examples are merely provided to help those skilled in the art to understand the essence of the present invention, and do not limit the protection scope of the present invention.

What is claimed is:

1. A method of preparing a test kit for predicting a short-term prognosis and a long-term prognosis of an acute myocardial infarction (AMI) patient undergoing percutaneous coronary intervention (PCI), comprising applying an S100A8/A9 complex in preparing the test kit; wherein,
the predicting the short-term prognosis and the long-term prognosis of the AMI patient undergoing the PCI refers to determining whether the AMI patient falls into a high-risk group of a postoperative adverse event (AE) or a low-risk group of the postoperative AE;
the postoperative AE comprises death, cardiogenic shock (CS), and acute heart failure (AHF); and
the S100A8/A9 complex is a heterodimer complex formed by S100A8 protein (Calgranulin A, MRP8) and S100A9 protein (Calgranulin B, MRP14) in a calcium ion-dependent manner.

2. The method according to claim 1, wherein,
the S100A8/A9 complex refers to the S100A8/A9 complex in serum of the AMI patient within 24 h after the PCI.

3. The method according to claim 2, wherein,
the short-term prognosis and the long-term prognosis refer to a prognosis during an intra-hospital period after the PCI and a prognosis 6 months or longer after the PCI, respectively.

4. The method according to claim 1, wherein,
(1) the high-risk group of the postoperative AE for the short-term prognosis has an AE incidence of 68% after the PCI, and the low-risk group of the postoperative AE for the short-term prognosis has an AE incidence of 21% after the PCI; and
(2) the high-risk group of the postoperative AE for the long-term prognosis has an AE incidence of 23% after the PCI, and the low-risk group of the postoperative AE for the long-term prognosis has an AE incidence of 6% after the PCI.

5. The method according to claim 1, wherein,
the high-risk group of the postoperative AE is determined when a serum S100A8/A9 complex expression level in the AMI patient 24 h after the PCI is greater than or equal to 4,540 ng/ml; and
the low-risk group of the postoperative AE is determined when the serum S100A8/A9 complex expression level in the AMI patient 24 h after the PCI is less than 4,540 ng/ml.

6. The method according to claim 1, wherein the test kit comprises a detection reagent for detecting a serum S100A8/A9 complex expression level, and the detection reagent comprises:
(1) an antibody specifically binding to the S100A8/A9 complex, wherein the antibody comprises a polyclonal antibody (pAb), a monoclonal antibody (mAb), a single-chain variable fragment (scFv), a functional antibody fragment, an antibody Fab region, a nanobody, a chimeric antibody, and a multispecific antibody;
(2) a ligandin or polypeptides specifically binding to the S100A8/A9 complex; and
(3) a non-protein compound specifically recognizing the S100A8/A9 complex.

7. The method according to claim 6, wherein the test kit is one selected from the group consisting of
(1) an enzyme-linked immunosorbent assay (ELISA) kit;
(2) a colloidal gold test strip assay kit;
(3) a chemiluminescent assay kit; and
(4) a flow cytometry (FCM) assay kit.

8. (canceled)

9. A method of a prediction of a short-term prognosis and a long-term prognosis of an AMI patient undergoing PCI, comprising applying an S100A8/A9 complex in the prediction: wherein,
the prediction of the short-term prognosis and the long-term prognosis of the AMI patient undergoing the PCI refers to determining whether the AMI patient falls into a high-risk group of a postoperative AE (short-term and long-term) or a low-risk group of the postoperative AE (short-term and long-term);
the postoperative AE comprises death, CS, and AHF;
the S100A8/A9 complex refers to the S100A8/A9 complex in serum of the AMI patient within 24 h after the PCI; and
the S100A8/A9 complex is a heterodimer complex formed by S100A8 protein (Calgranulin A, MRP8) and S100A9 protein (Calgranulin B, MRP14) in a calcium ion-dependent manner.

10. The method according to claim 9, wherein,
the short-term prognosis and the long-term prognosis refer to a prognosis during an intra-hospital period after the PCI and a prognosis 6 months or longer after the PCI, respectively;
the high-risk group of the postoperative AE for the short-term prognosis has an AE incidence of 68% after the PCI;
the low-risk group of the short-term prognosis has an AE incidence of 21% after the PCI;
the high-risk group of the postoperative AE for the long-term prognosis has an AE incidence of 23% after the PCI;
the low-risk group of the postoperative AE for the long-term prognosis has an AE incidence of 6% after the PCI;
the high-risk group of the postoperative AE (short-term and long-term) is determined when a serum S100A8/A9 complex expression level in the AMI patient 24 h after the PCI is greater than or equal to 4,540 ng/ml; and
the low-risk group of the postoperative AE (short-term and long-term) is determined when the serum S100A8/A9 complex expression level in the AMI patient 24 h after the PCI is less than 4,540 ng/ml.

11. The method according to claim 2, wherein,
(1) the high-risk group of the postoperative AE for the short-term prognosis has an AE incidence of 68% after the PCI, and the low-risk group of the postoperative AE for the short-term prognosis has an AE incidence of 21% after the PCI; and
(2) the high-risk group of the postoperative AE for the long-term prognosis has an AE incidence of 23% after the PCI, and the low-risk group of the postoperative AE for the long-term prognosis has an AE incidence of 6% after the PCI.

12. The method according to claim 3, wherein,
(1) the high-risk group of the postoperative AE for the short-term prognosis has an AE incidence of 68% after the PCI, and the low-risk group of the postoperative AE for the short-term prognosis has an AE incidence of 21% after the PCI; and
(2) the high-risk group of the postoperative AE for the long-term prognosis has an AE incidence of 23% after the PCI, and the low-risk group of the postoperative AE for the long-term prognosis has an AE incidence of 6% after the PCI.

13. The method according to claim 2, wherein,
the high-risk group of the postoperative AE is determined when a serum S100A8/A9 complex expression level in the AMI patient 24 h after the PCI is greater than or equal to 4,540 ng/ml; and
the low-risk group of the postoperative AE is determined when the serum S100A8/A9 complex expression level in the AMI patient 24 h after the PCI is less than 4,540 ng/ml.

14. The method according to claim 3, wherein,
the high-risk group of the postoperative AE is determined when a serum S100A8/A9 complex expression level in the AMI patient 24 h after the PCI is greater than or equal to 4,540 ng/ml; and
the low-risk group of the postoperative AE is determined when the serum S100A8/A9 complex expression level in the AMI patient 24 h after the PCI is less than 4,540 ng/ml.

15. The method according to claim 2, wherein the test kit comprises a detection reagent for detecting a serum S100A8/A9 complex expression level, and the detection reagent comprises:
(1) an antibody specifically binding to the S100A8/A9 complex, wherein the antibody comprises a polyclonal antibody (pAb), a monoclonal antibody (mAb), a single-chain variable fragment (scFv), a functional antibody fragment, an antibody Fab region, a nanobody, a chimeric antibody, and a multispecific antibody;
(2) a ligandin or polypeptides specifically binding to the S100A8/A9 complex; and
(3) a non-protein compound specifically recognizing the S100A8/A9 complex.

16. The method according to claim 3, wherein the test kit comprises a detection reagent for detecting a serum S100A8/A9 complex expression level, and the detection reagent comprises:
(1) an antibody specifically binding to the S100A8/A9 complex, wherein the antibody comprises a polyclonal antibody (pAb), a monoclonal antibody (mAb), a single-chain variable fragment (scFv), a functional antibody fragment, an antibody Fab region, a nanobody, a chimeric antibody, and a multispecific antibody;
(2) a ligandin or polypeptides specifically binding to the S100A8/A9 complex; and
(3) a non-protein compound specifically recognizing the S100A8/A9 complex.
